(19) United States
(12) Patent Application Publication
(10) Pub. No.: US 2004/0142399 A1
(43) Pub. Date: Jul. 22, 2004

(54) Recombinant FlaA as a diagnostic reagent

(76) Inventors: Robert D. Gilmore JR., Fort Collins, CO (US); Barbara JB Johnson, Fort Collins, CO (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/675,447

(22) Filed: Sep. 30, 2003

Related U.S. Application Data
(63) Continuation of application No. 09/004,395, filed on Jan. 8, 1998.

(51) Int. Cl. C07K 14/20; G01N 33/554; G01N 33/569; C07H 21/04

(52) U.S. Cl. 435/7.32; 435/69.3; 435/252.3; 435/320.1; 530/350; 536/23.7

(57) ABSTRACT

The present invention provides for compositions and methods for serological immunoassay for the detection of Lyme disease using recombinant P37/FlaA protein antigen and methods for producing such protein antigen.

### STATEMENT OF GOVERNMENT RIGHTS

This work has been supported in whole or in part by the Department of Heath & Human Services, Centers for Disease Control and Prevention (CDC), National Center for Infectious Diseases, thus the Government of the United States of America may have certain rights in the present invention.

### FIELD OF THE INVENTION

This invention relates to the field of diagnostic assays for Lyme disease. In particular, the invention relates to reagents and methods for diagnostic assays, and automated diagnostic assays.

### BACKGROUND OF THE INVENTION

Lyme disease (Ld) is a multi-system disorder caused by the spirochetes of Borrelia burgdorferi sensu lato complex which are transmitted by Ixodes ticks. It is the most commonly reported arthropod-borne human infection in the United States (Lyme Disease—United States, 1996. MMWR Morb. Mortal. Wkly. Rep. 1997, 46(23):531-535). Lyme disease was first recognized in the United States in 1975 when an unusual cluster of childhood arthritis cases appeared in Lyme, Connecticut (Steere A C et al., “Lyme arthritis; an epidemic of oligoarticular arthritis in children and adults in three Connecticut communities.” Arthritis Rheum. 1977, 20(1):7-17). Epidemiological evidence associated these cases with the bite of ticks (Steere A C et al., “Erythemea chronicum migrans and Lyme arthritis: epidemiologic evidence for a tick vector.” Am J. Epidemiol. 1978, 108(4): 312-321), later identified as Ixodes scapularis (Burgdorfer W et al., “Lyme disease-a tick borne spirochetosis?” Science 1982, 216(4552): 1317-1319; Oliver J H et al., “Conspecificity of the ticks Ixodes scapularis and I dammini (Acari: Ixodidae)” J Med Entomol. 1993, 30(1): 54-63). Early Lyme disease is characterized by an expanding lesion, erythema migrans (EM). headache. fever and mylagia. Muscoskeletal, cardiac, skin, and neurological disorders can follow weeks to months later (Steere A C, “Lyme disease” N Engl. J. Med. 1989, 321(9): 586-596).

Correct early diagnosis of Lyme disease is important since prompt adequate antibiotic therapy can prevent the serious manifestations of long-term infection (Steere A C et al., “Antibiotic therapy in Lyme Disease.” Ann. Inteml. Med. 1980, 93(1): 1-8). Clinical diagnosis can be assisted by serologic tests, provided they have high sensitivity and specificity. (Tugwell P et al., 1997, “Laboratory evaluation in the diagnosis of Lyme disease” Ann. Intern. Med. 127: 1109-1123). The present invention advances the art of serodiagnosis of Lyme disease, particularly in persons with recently acquired infection.

Historically, the first serologic tests used were indirect immunoflourescence assays (IFA) that employed fixed whole spirochetes (Steere A C et al., “The spirochetal etiology of Lyme disease.” N. Eng. J. Med. 1983, 308(13): 733-740). Shortly thereafter, the first immunoblots were done to identify immunodominant antigens of B. burgdorferi (Barbour A G et al., “Antibodies of patients with Lyme disease to components of the Ixodes dammini spirochete.” J. Clin. Invest. 1983, 72(2): 504-515). It has become increasingly recognized that some antigens are differentially expressed in the vector tick and mammalian hosts. Some antigens, such as OspC, are up-regulated in the course of tick feeding and are important antigens in the early immune response to infection (Schwann T G et al., “Induction of an outer surface protein on Borrelia burgdorferi during tick feeding.” Proc. Natl. Acad. Sci. (USA), 1995, 92(7): 2909-2913). OspA and OspB are down regulated and have limited serodiagnostic utility in early Lyme disease. Some antigens are expressed exclusively in infected mammals (Fikrig E et al., “Borrelia burgdorferi P35 and P37 proteins, expressed in vivo, elicit protective immunity.” Immunity, 1997, 6(5):531-539).

The best choice of defined antigens to be incorporated into a serodiagnostic test is the subject of active research. At the present time, most commercial serum-based assays use a lysate of whole B. burgdorferi in an enzyyme-linked immunoassay (EIA). These EIAs detect antibodies that are elicited in the course of other diseases, both infections and autoimmune, as well as antibodies specific for determinants of B. burgdorferi (Hansen K et al., “Immunochemical characterization of and isolation of the gene for a Borrelia burgdorferi immunodominant 60-kilodalton antigen common for a wide range of bacteria.” Infect. Immun., 1988, 56(8): 2047-2053; Dressler F et al., “Western blotting in the serodiagnosis of Lyme disease.” J. Infect. Dis., 1993, 167(2): 392-400). As a consequence, EIAs based on whole cell antigens have inadequate specificity.

In recognition of the aforementioned specificity problem, the Association of State and Territorial Public Health Laboratory Directors (ASTPLD) and the Centers for Disease Control and Prevention (CDC) have recommended that a two-step approach to serodiagnosis be used until simple tests with better performance characteristics have been developed. The first step is a sensitive EIA or IFA. Samples scored positive or borderline are then subjected to a test with increased specificity, a Western blot (ASTPHLD Recommendations. In: “Proceedings of the Second National Conference on Serologic Diagnosis of Lyme disease.” 1995, pp. 1-7; Johnson B J et al., “Serodiagnosis of Lyme disease: accuracy of a two-step approach using a flagella-based ELISA and immunoblotting.” J. Infect. Dis. 1996, 174(2): 346-353).

A number of different sets of criteria have been advocated for the interpretation of Western blots. Interim guidelines recommended by ASTPHLD and CDC are that the criteria of Dressler et al. (1993, supra), be used to interpret IgG blots and the criteria of Engstrom et al., (Engstrom S M et al., “Immunoblot interpretation criteria for serodiagnosis of early Lyme disease.” J. Clin. Microbiol., 1995, 33(2): 419-427), be applied to IgM blots.

One line of new research that was advocated at the time that the interim guidelines were adopted was to study an antigen of 37 kDa that is expressed in cultured spirochetes (Dressler et al., 1993 supra; ASTPHLD 1995 supra; Aguero-Rosenfeld M E et al., “Evolution of the serologic response to Borrelia burgdorferi in treated patients with culture-confirmed erythema migrans.” J. Clin. Microbiol., 1996, 34(1): 1-9). However, the 37 kDa antigen (“P37”) was not included in the guidelines for scoring IgM immunoblots because the antigen had not been definitively identified by molecular tools, and neither antibody nor antigen markers were available for it (ASTPHLD 1995 supra). The ASTPHLD/CDC recommendations were that P37 could be included in the scoring criteria for IgM blots once calibration antibodies were available to this protein.

The present invention describes the definitive identification of the serodiagnostic antigen referred to as P37 as FlaA, an outer sheath protein of the periplasmic flagella of B. burgdorferi (Ge and Charon, “An unexpected flaA homolog is present and expressed in Borrelia burgdorferi.” J. Bacteriol. 1997, 179(2):552-6). The antigen that comprises the core of the flagellar filaments is a 41 kDa protein known as FlaB (Barbour A G et al., “A Borrelia specific monoclonal antibody binds to a flagellar epitope.” Infect. Immun., 1986, 52(2): 549-554). A previous report had failed to find that FlaA was useful for the serodiagnosis of Lyme disease (Ge and Charon, “A putative flagellar outer sheath protein is not an immunodominant antigen associated with Lyme disease.” Infect. Immun., 1997, 65(7): 2992-2995). Here we show, contrary to the prior teaching in the art, that the methods and compositions of the instant invention demonstrate that FlaA is indeed a prominent antigen in the early humoral immune response to B. burgdoferi infection, and significantly suitable for use in improved serologic tests for exposure to Lyme disease spirochetes.

Various patents have described methods and reagents for diagnosis and treatment of Lyme disease, including the following U.S. patents (all of which are hereby incorporated by reference in their entirety):

U.S. Pat. No. 5,523,089 issued Jun. 4, 1996 entitled “Borrelia Antigen” which describes the B fraction of B. burgdorferi, methods for preparing the B fraction, and compositions containing the B fraction, which is substantially free of cell wall and flagellar components.

U.S. Pat. No. 5,554,371 issued Sep. 10, 1996 entitled “Recombinant Vaccine Against Lyme Disease” which describes a highly-antigenic, recombinant polypeptide of a molecular weight of about 110 kDa which is antigenically distinct from the OspA, OspB and 41 kDa flagellin proteins.

U.S. Pat. No. 5,558,993 issued Sep. 24, 1996 entitled “Cloned B. burgdorferi Virulence Protein” which describes a polynucleotide encoding a 17 kDa virulence protein called EppA.

U.S. Pat. No. 4,888,276 issued Dec. 19, 1989 entitled “Method and Composition for the Diagnosis of Lyme Disease” which describes a non-invasive assay for detecting lyme disease antigens of 31 kDa and 34 kDa molecular weight.

Thus it well recognized that it would be useful to have reliable assay means for detecting exposure to Lyme disease spirochete that are specific for B. burgdorferi.

### SUMMARY OF THE INVENTION

The present invention describes a recombinant protein antigen encoded for by the nucleic acid sequence of SEQ ID NO.:1, which comprises the amino acid sequence of SEQ ID NO.:2 and the use of the recombinant protein antigen for bioassays to detect early Lyme disease. In a preferred embodiment the recombinant protein antigen is the FlaA gene product, which is now identified as the P37 protein, and has the amino acid sequence of SEQ ID NO.:2 without the signal peptide.

The present invention provides for an assay for detecting Lyme disease infection comprising obtaining a serum sample from a patient to be tested, contacting said serum sample with recombinant P37 (37 kDa) protein, and detecting any antibody specifically bound to said protein. In a preferred embodiment the P37/FlaA protein antigen has the amino acid sequence of SEQ ID NO.:2, and in a most preferred embodiment it is lacking the signal peptide. In a preferred embodiment, the antibody detected is of the IgM subclass. In a preferred embodiment the recombinant P37/FlaA protein antigen is produced as a fusion protein, such that the fusion partner does not interfere with the antigenic epitope/s of the P37/FlaA protein antigen. A preferred fusion partner is the approximately 38 kDa T7 gene 10 product.

The present invention encompasses manually performed assays as well as automated assays. The assay of the present invention can be designed to directly detect antibodies in test samples which will specifically bind to recombinant P37 protein, wherein the antibodies to be detected are labeled by derivatized secondary binding protein. The assay of the invention can be designed with the recombinant P37 antigen immobilized on a solid support or in solution. Depending upon assay format, antibodies from the sample to be tested may be isolated by specific binding to recombinant P37 antigen, and the identification of the specific antibodies are made by derivatized secondary binding protein, or any such suitable detection means.

In an alternative format, the recombinant P37 protein antigen can be labeled with a detectable tag, such that antibodies in the test sample which will specifically bind the recombinant P37 protein antigen can be labeled by the bound P37. In such an assay format, the antibodies of the test sample can be captured by binding protein and then assayed for specific binding to recombinant P37 protein antigen.

One of ordinary skill in the art would recognize that the demonstration that recombinant P37 is a suitable test antigen for the diagnosis of early Lyme disease allows for the design of several assay formats, labeling and detection schemes which are known in the art.

The present invention also provides for methods for the production of recombinant P37/FlaA protein antigen, wherein the method for producing recombinant FlaA protein from transformed cell cultures comprises constructing a DNA expression vector, containing an expressible FlaA encoding DNA sequence, transforming a suitable host cell with said expression vector, preparing large-scale cell cultures from fresh transformants of said host cell with said expression vector, and not overnight starter cultures, inducing FlaA protein expression from said large-scale cultures, and isolating recombinant FlaA protein. In a preferred embodiment the P37/FlaA protein antigen is produced as a fusion protein, such that the fusion protein partner does not interfere with the antigen epitope/s of the FlaA protein and subsequent serological recognition of the antigen.

### BRIEF DESCRIPTION OF THE DRAWINGS

The present invention is more clearly understood by reference to the following drawings in which:

FIG. 1 are diagrams of the expression constructs and primers.

FIG. 2A is a Protein gel showing expression of recombinant P37, and FIG. 2B are Western Blots showing the expression of recombinant P37 protein.

FIG. 3 are Western Blots showing the reactivity of recombinant P37 protein with Lyme patient and control serum.

FIG. 4 are Western Blots showing the use of recombinant P37 to detect reactivity in the serum of patients.

### BRIEF DESCRIPTION OF THE INVENTION

Serum samples submitted for Lyme disease testing are presently evaluated in a two-step process as recommended by the 2^{nd} National Conference on Serologic Diagnosis of Lyme Disease (ASTPHLD, 1995, “Association of State and Territorial Public Health Laboratory Directors and the Centers for Disease Control and Prevention, 1995, Recommendations,”, in Proceedings of the Second National Conference on Serologic diagnosis of Lyme Disease, (Dearborn, Mich., ASTPHLD, Washington, D.C.) p. 1-7).

The first test to be used is a sensitive serological assay such as an ELISA. All samples found to be equivocal or positive are then further tested by a more standardized Western blot procedure. Certain criteria are recommended in the interpretation of Western blot results. For serodiagnosis of early Lyme disease, IgM immunoblots are considered positive according to the criteria proposed by Engstrom et al., (1995, J. Clin. Micro. 33:419-427) i.e. if two of the following three bands are present: OspC (24 kDa), BmpA (39 kDa), and Fla (41 kDa). Recognition of a 37 kDa band (P37) was found to be significant in early Lyme disease immunoblots (Aguero-Rosenfeld et al., 1996, J. Clin. Micro. 34:1-9), but since there were no monoclonal antibodies or recombinant protein antigen markers available for P37, it was not included in the interpretation criteria (ASTPHLD, 1995, supra).

In view of the expected eventual molecular characterization of the P37 gene, to which monoclonal antibody and recombinant antigen could be produced, the recommendation was to eventually include P37 in the immunoblot criteria for IgM serology (ASTPHLD, 1995, supra).

As discussed above, it was long thought that B. burgdorferi expressed a single flagellin protein of 41 kDa, termed Fla. The Fla protein has been a prominent antigen for detection of Lyme disease infection, but is a highly cross-reactive antigen of many spirochetes. Recently, it has been discovered that B. burgdorferi periplasmic flagella (PFs) have more than one flagellin protein, similar to the PFs of most other spirochetes, which comprise an outer sheath of FlaA proteins, and a core filament of FlaB proteins. Analysis of B. burgdorferi species 212 showed that there was a flaA gene homolog with a deduced polypeptide having 54 to 58% similarity to FlaA from other spirochetes upstream from the cheA gene. Immunoblots using anti-FlaA serum from Treponema pallidum on a lysate of B. burgdorferi showed strong reactivity to a protein of 38.0 kDa, consistent with expression of flaA in growing cells (Ge and Charon, 1997, J. Bacteriology 179(2):552-556).

Thus the previously known 41 kDa flagellin protein Fla corresponds to the FlaB core filament proteins of other spirochetes. Ge and Charon attempted to generate recombinant FlaA protein, and used various expression vector systems because it was known that overexpression of T. pallidum FlaA in E. coli was toxic to the host cell. Expecting that overexpression of B. burgdorferi Fla would be difficult, several flaA constructs were tested using different expression systems, including pPROEX-1, pMAL-p2, pGXT-2T, and pET-23a. As a host cell for transformation and expression of protein, E. coli BL21(DE3) plysE was used to express flaA and its derivatives under the T7 promoter (pET-23a), and E. coli DH5′α was utilized in the cloning and expression of the other recombinant FlaA proteins. Fusion proteins were also generated: His fusion protein for purification by Ni-NTA resin, maltose binding fusion protein by amylose resin, and glutathione S-transferase fusion protein by glutathione agarose. All attempts to express intact FlaA protein with a complete N-terminal signal sequence resulted in failure. It was determined that two forms of truncated FlaA, lacking amino acids 1 to 26 of the signal sequence, and lacking amino acids 1 to 76, could be expressed in E. coli. (Ge and Charon, 1997, Infec. Immunity 65(7):2992-2995). However, the authors concluded that although FlaA is a protein unique to spirochetes, it is not a good candidate for serodiagnosis of Lyme disease.

Here we describe the isolation and cloning of the P37 gene, the recognition that this is in fact flaA, the production of recombinant P37 protein, the serologic reactivity of Lyme disease patient serum samples with the recombinant P37, and the successful use of recombinant P37 in diagnostic assay for Lyme disease.

The present invention will be better understood and exemplified by reference to the following examples which are meant by way of illustration and not as a limitation. One of ordinary skill in the art will recognize that certain modifications and adaptations of the teaching of the present invention may be made without undue experimentation, without departing from the spirit and scope of the present invention.

### EXAMPLE 1

### Isolation and Identification of a P37 Gene Clone

A genomic DNA library of B. burgdorferi strain B31 (low passage) was constructed in the phage lambda vector, ZapExpress™ (Stratagene, La Jolla, Calif., USA) as follows. Total DNA was purified from cultured B. burgdorferi cells as described in Gilmore et al. (Gilmore et al., “Outer surface protein C (OspC), but not P39, is a protective immunogen against a tick-transmitted Borrelia burgdorferi challenge: evidence for a conformational protective epitope in OspC.” Infect. Immun., 1996, 64:2234-2239). The DNA was subjected to a partial Sau3A restriction enzyme digestion to generate fragments ranging in size from approximately 1 kb to 10 kb. The digested DNA fragments were ligated into BamH1 cut Lambda ZapExpress™, and packaged according to the manufacturer's directions. The phage library was plated onto E. coli host cells XL1-Blue MRF (Stratagene), amplified, titred, and stored at 4° C.

The B. burgdorferi genomic lambda expression library was screened immunologically using a polyclonal anti-P37 antibody obtained from the serum of a Lyme disease patient with a strong IaG response to P37 as seen on Western blots. The patient serum was immunoblotted against B. burgdorferi proteins electrophoretically separated and transferred to nitrocellulose membranes according to standard procedures. (For standard protocols and methods see generally Sambrook et al., 1989 Molecular Cloning, 2^{nd} edition, Cold Spring Harbor Press; Ausubel et al., 1992 Short Protocols in Molceular Biology, 2^{nd} edition, John Wiley & Son; Rose et al., 1986 Manual of Clinical Laboratory Immunology, 3^{rd} edition, American Society of Microbiologists; Gene Expression Technology 1991. Methods in Enzymology Vol. 185). This antiserum was immunoblotted against B. burgdorferi whole cell lysate antigens, visualized using alkaline phosphatase-conjugated secondary antibody, and developed with the substrates 5-bromo-4chloro-3-indolyl-phosphate (BCIP) and nitroblue tetrazolium (NBT). During calorimetric visualization of the immunoreactive bands, the reaction was stopped by removal of the substrate and a subsequent rinse in wash buffer (10 mM Tris-HCl pH 7.5, 0.5% Tween-20, 0.9% NaCl). The nitrocellulose containing the detected P37 band was excised and minced into 2 mm pieces using a clean scalpel and placed into a microfuge tube. Glycine (0.4 ml of 100 mM, pH 2.8) was added, and the tube vortexed lightly for approximately 1 minute. The clycine solution was removed, and the procedure repeated twice more. The solution was neutralized by the addition of 0.15 ml of 1 M Tris pH 8.8. An equal volume of 5% skim milk in wash buffer was added to the eluted antibody mixture, which was stored at 4° C. This served to provide a monospecific anti-P37 antibody pool for use in the screening of the lambda genomic library.

Phage from the B. burgdorferi genomic library were plated and probed with the eluted P37-specific antibody according to procedures described in Gilmore et al., (supra). Positive antibody-reactive plaques were picked, plaque purified, and the phagemid pBK-CMV was rescued by the in vivo excision procedure provided by the manufacturer (Stratagene). Resultant colonies were grown in culture, and recombinant protein expression was induced by addition of isopropyl-1-thio-β-D-galactopyranoside (IPTG) to 0.5 mM. Cell pellets were harvested, subjected to protein fractionation by sodium dodecyl sulfate polyacrylamide gel electrophoresis (SDS-PAGE), and proteins were electrophoretically transferred to nitrocellulose or polyvinylidene difluoride (PVDF) membranes (Schleicher & Schuell, Keane, N.H. USA) according to standard procedures. Transferred proteins from the recombinant E. coli lysate were immunoblotted against the eluted anti-P37 antibodies.

Screening the B. burgdorferi genomic library with P37 monospecific antibody yielded positive clones, which were selected and further analyzed for recombinant protein expression by Western blot. A truncated recombinant product of about 34 kDa with reactivity with the anti-P37 antibodies was observed. Plasmid DNA was isolated from this clone and subjected to DNA sequence analysis. By using pBK-CMV vector-specific primers, sequence data were generated from both ends of the cloned insert. The approximately 450 bp of DNA sequence obtained from one end of the insert was searched against the GenBank database using the Basic Local Alignment Search Tool (BLAST) program. The alignment search resulted in an exact match of the query sequence to that of the flaA gene of B. burgdorferi strain 212 (accession number U62900). The DNA sequence from the opposite end of the insert showed an alignment similarity match to a chemotaxis gene, cheW of B. burgdorferi, albeit not an exact match. A motility-chemotaxis operon in B. burgdorferi 212 consisting of the flaA gene and five chemotaxis genes was recently described (Ge and Charon, 1997, “Molecular characterization of a flagellar/chemotaxis operon in the spirochete B. burdorferi.” FEMS Microbiol. Letters 153: 425-431; Ge and Charon, 1997, “An unexpected flaA homolog is present and expressed in Borrelia burgdorferi” J. Bacteriol. 179: 552-556). The truncated flaA sequence of our insert was in frame with the lacZ fusion partner of the pBK-CMV expression vector and was inducible by IPTG, therefore suggesting that the identity of the expressed recombinant product was FlaA.

Plasmid DNA containing the cloned P37 gene insert was purified and sequenced using standard techniques. Recombinant plasmid DNA was isolated from E. coli using a QIAprep-spin Plasmid Kit (Qiagen, Chatsworth, Calif., USA), according to the manufacturer's directions. DNA sequencing was performed with the Taq DyeDeoxy Terminator Cycle Sequencing Kit (Applied Biosystems, Inc., Foster City, Calif., USA) according to the manufacturer's directions. Sequencing reactions were run and analyzed by the automated sequencing apparatus Model 373A (Applied Biosystems). DNA sequences were analyzed with Lasergene software (DNASTAR, Madison, Wis., USA).

### EXAMPLE 2

### Expression of Recombinant FlaA in E. coli

To prove that the recombinant product was FlaA, the flaA gene was subcloned into an E. coli expression vector, expressed, and tested against the anti-P37 antibodies.

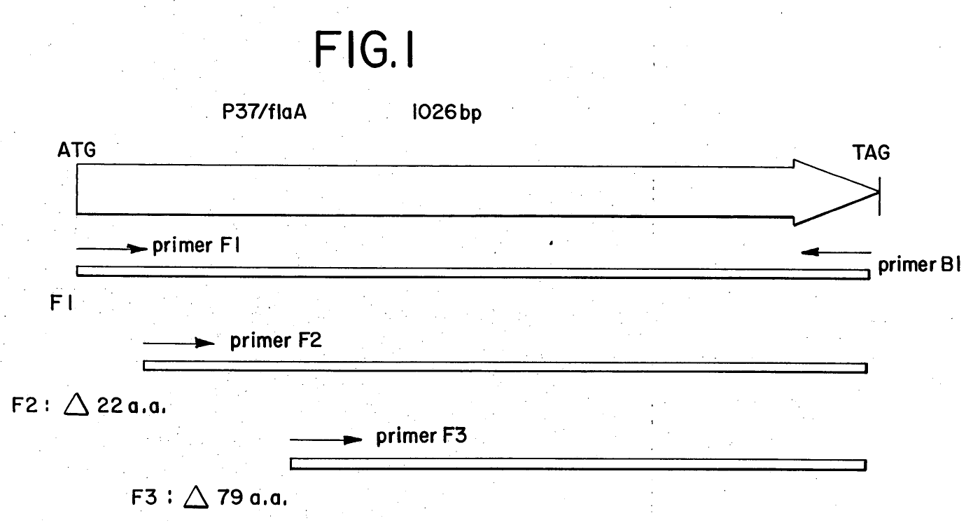

Three constructs of the P37 gene coding sequence were generated by PCR amplification of B. burdorferi genomic DNA. Construct F1 constituted the entire coding sequence. Construct F2 constituted the entire coding sequence minus the leader peptide (the first 22 amino acids); and construct F3 constituted the coding sequence beginning at amino acid 80, which corresponded to the original cloned insert. FIG. 1 illustrates the orientation of the PCR primers and the construct DNA sequences.

The original P37 clone described above did not express a full-length protein, and the DNA sequence revealed that the gene was truncated; i.e. did not have the full amino-terminus. The GenBank FlaA sequence entry was used to determine that the first 79 amino acids of the protein were missing from the original clone. From the DNA sequence information, we were able to construct primers for PCR amplification. The complete B. burgdorferi FlaA coding sequence was amplified from genomic DNA using the primers described in FIG. 1 by standard PCR methods. Primer F1 was the nucleic acid sequence 5′-ATGAAAAGGAAAGCTAAAAGT-3′ (SEQ ID NO.:3); primer F2 was the nucleic acid sequence 5′-GATGGATTAGCAGAGGGTT-3′ (SEQ ID NO.:4); primer F3 was the nucleic acid sequence 5′-TGGGATAAATAATTGGAGCGT-3′ (SEQ ID NO.:5); and the reverse primer for all reactions was the primer B1 having the nucleic acid sequence 5′-CTAATTTTTCGGAGATGATTC-3′ (SEQ ID NO.:6). PCR reactions were preformed with approximately 1 μg template DNA, and the parameters were 35 cycles at 94° C. for 30 seconds, 45° C. for 30 seconds, 72° C. for 2 minutes using a GeneAmp™ PCR System 9600 (Perkin Elmer, Norwalk, Conn., USA).

The amplified coding sequence fragments were ligated into the plasmid expression vector pSCREEN-1b (Novagen, Madison, Wis., USA), a pET vector derivative, and transformed into E. coli cells NovaBlue (DE3) according to standard procedures and the manufacturer's directions (construct F1). The transformation mixture was plated onto Luria-Bertani (LB) plates containing 0.25 mg/ml carbenicillin. Two other constructs were made in a similar manner. One eliminated the putative signal peptide (by deleting the first 22 amino acids; construct F2), and the other began at amino acid 80, as in the original P37 clone (construct F3).

A primary culture for expression was started in LB broth containing 0.25 mg/ml carbenicillin by inoculating with a colony from a fresh transformant plate as described above. The culture was incubated at 37° C. with shaking, and observed. When the cells had grown to approximately mid-logarithmic stage (ie. O.D.₆₀₀ of around 0.6), IPTG was added at a concentration of 0.5-1.0 mM to induce protein expression. Cultures were allowed to grow for approximately 2-3 hours following induction. Aliquots of cells were pelleted and suspended in SDS-PAGE loading buffer, boiled for 5 minutes, and run on SDS-PAGE according to standard procedures. Following electrophoresis, the gel was transferred to a membrane and immunoblotted with the anti-P37.

SDS-PAGE analysis showed that the construct F2 was the only one of the three constructs which expressed recombinant P37. The recombinant P37 protein is expressed as a fusion protein with the partner being the approximately 38 kDa T7 gene 10 product from the pSCREEN vector. Confirmation of the recombinant protein was done in a Coomassie Blue stained gel.

In this expression system, the recombinant gene was in frame with the vector-encoded T7 gene product of about 38 kDa, which resulted in a recombinant fusion product.

Unexpectedly, we have discovered that for successful expression of protein, it was essential to start the expression culture with a fresh transformant colony, and not subculture from an overnight starter culture. Cells propagated from a subculture produced little or no recombinant protein in this expression system. Only our method for inoculation of a primary culture for protein expression from a fresh transformant colony resulted in the satisfactory expression of recoverable protein. Expression of protein following this method could yield from about 10 to 100 mg/L protein product.

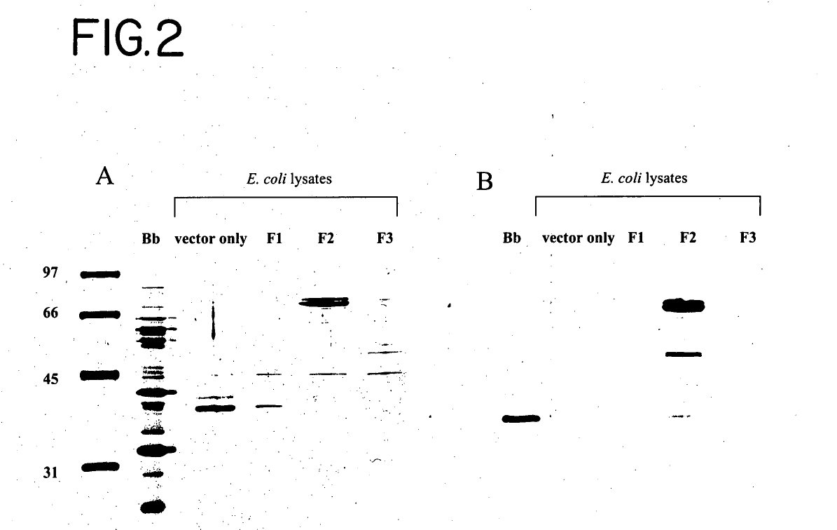

FIG. 2A shows the protein profile of the constructs and their ability to express FlaA, with the corresponding Western blot in FIG. 2B. Constructs F1 and F3 did not express any recombinant protein reactive with the anti-P37 antibodies. Construct F2 turned out to be the most stable of the three, as it expressed an approximately 75 kDa fusion product as predicted. The Western blot in FIG. 2B shows the recombinant product was reactive to the anti-P37 antibody, indicating that FlaA and P37 are the same protein.

### EXAMPLE 3

### Lyme Patient Serum Samples

Early Lyme disease patient serum samples demonstrated to have IgM reactivity against P37 in Western blots were tested for their reactivity against the recombinant P37 antigen. Lyme disease case serum samples were obtained from patients with erythema migrans (EM, n=40) residing in the endemic areas of New York, Wisconsin, and New England. All samples were acute-phase specimens obtained on the day that the patient was first seen by a physician, before antibiotic therapy was begun (baseline samples). The clinical diagnosis of EM was supported by culture isolation of B. burgdorferi from a skin biopsy specimen in 65% of the patients; isolation was not attempted in the other cases. All physicians who provided serum samples had extensive experience in the clinical diagnosis of Lyme disease. Samples were classified as being from persons with primary infections (a single EM, n=23) or disseminated disease (multiple EMs, n=17). The length of time from onset of symptoms to venipuncture was about 3 days longer for patients with multiple EMs compared with patients with single rashes (median of 8 days vs. 5; mean of 10.2 days vs. 6.5).

Potentially cross-reactive serum samples were from syphilis patients residing in Texas. The syphilis serum samples had reciprocal end-point titers in the VDRL test of 2, 2, 16, and 64. Negative control serum samples were from healthy blood donors residing in an area non-endemic for Lyme disease (Atlanta, Ga. USA).

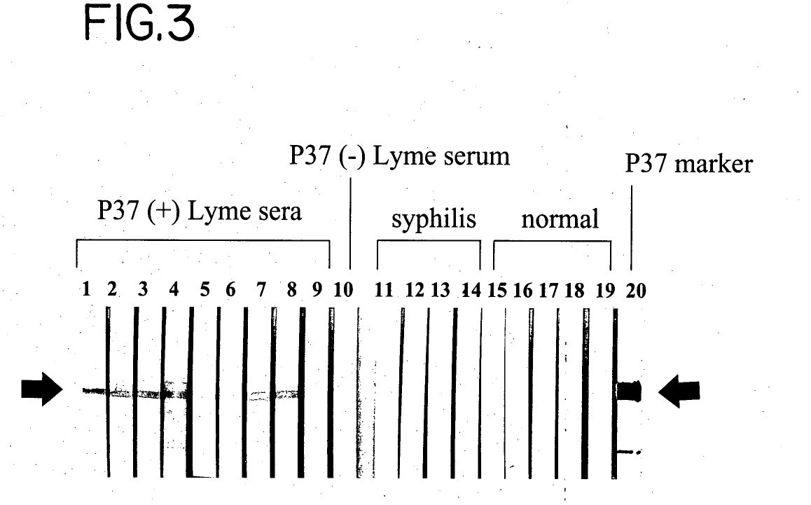

In an initial study, nine P37-positive Lyme disease patient samples were assayed. These serum samples were pre-selected for IgM antibodies to the B. burgdorferi P37 antigen by immunoblotting. These samples were blotted against the recombinant antigen made from the F2 construct, together with ten controls (one anti-P37 negative sample from a patient with EM, four syphilis serum samples, and five samples from healthy blood donors). FIG. 3 shows that all nine P37-positive Lyme disease patients were reactive with the recombinant P37 antigen, while all other samples failed to show appreciable immunoreactivity. Although there is some background reactivity seen with the recombinant P37 antigen fusion protein in the negative serum samples, the positive samples were clearly distinguishable from these. They also showed no reactivity to the fusion partner alone when immunoblotted against E. coli lysate containing plasmid vector only.

Next, a survey was done to assess the immunoreactivity of serum from patients with early Lyme disease with the recombinant P37 antigen. Forty additional samples, i.e. not including the ones screened in FIG. 3, were assayed by immunoblotting against both recombinant P37 antigen and B. borderferi proteins. In addition to a positive control serum, there were four negative samples assayed to assess background reactivity. Any sample showing reactivity equal to that of negative serum was considered negative.

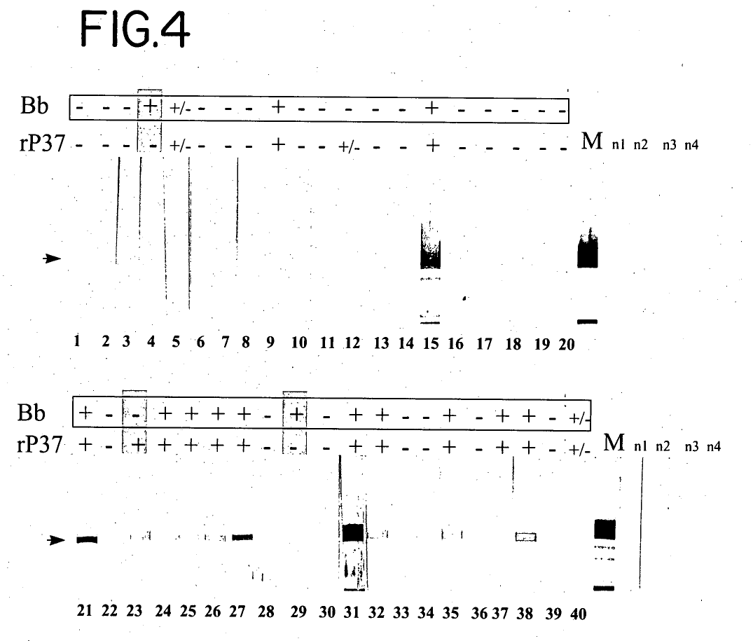

The results are shown in FIG. 4. Of the 40 samples tested, 13 were positive with recombinant P37, 24 were negative, and 3 were scored as plus. minus (+/−) indicative of a weakly positive or negative reaction. When compared with the B. burgdorferi whole cell lysate assay, there were only three discrepancies. One sample positive for the recombinant P37 was negative on the whole cell protein blot, and conversely, two samples negative for the recombinant protein were positive on the whole cell blot. Of the total samples tested, i.e. FIGS. 3 and 4, there were 3 discrepancies out of 61 samples tested (5%). When compared to blots against B. burdorferi whole cell antigens, serum samples with a prominent anti-P37 signal gave equally strong signals against recombinant P37. Accordingly, samples that gave weak signals in B. Burgdorferi blots (and were classified as plus/minus for P37). were also weak against the recombinant P37.

The Western blot results demonstrate that recombinant P37 protein is useful and can be used in assays for the early detection of Lyme disease.

Immunoblotting of serum samples against B. burgdorferi antigens were performed at dilutions of 1:100 on MarBlot strips (MarDx, Carlsbad, Calif., USA) according to the manufacturer's directions. Immunoblotting of serum samples against recombinant P37 were performed by fractionating the induced E. coli lysate on 10% polyacrylamide gels, with subsequent transfer to nitrocellulose using a Mini Trans-Blot system (BioRad, Hercules, Calif. USA), with transfer buffer conditions being 25 mM Tris, 192 mM glycine, 20% w/v methanol, pH 8.3. Serum samples were blotted to the recombinant P37 antigen lysate at dilutions of 1:1000 for at least 1 hour. Following 3 wash buffer rinses in a total of 15 minutes, the blots were incubated with an anti-human IgM conjugated with alkaline phosphatase (Kirkegard & Perry, Gaithersburg, Md., USA) at 1:1000 for at least 30 minutes. The blots were developed with BCIP/NBT substrate.

IgM reactivity to P37 is prominent in the evolution of the early serologic response to B. burgdorferi in patients with EM (Dressler F, et al. “Western blotting in the serodiagnosis of Lyme disease” J. Infect. Dis. 167: 392-400). In a study by Aguero-Rosenfeld et al., the most frequent immunoblot bands were to OspC, FlaB, and P37 (Aguero-Rosenfeld M F et al., 1996, “Evolution of the serologic response to Borrelia burgdorferi in treated patients with culture-confirmed erythema migrans.” J. Clin. Microbiol. 34: 1-9). In persons with EM of ≧7 days duration at venipuncture (n=17), the frequency of IgM seroreactivity to P37 was 71%, compared with 76% to OspC and 82% to FlaB. In persons with very early disease (<7 days from onset of EM, n=29), this frequency was 14% compared with 48% to OspC and 31% to FlaB.

In a study from this laboratory, of 70 patients with EM (50/70 from persons in whom B. burgdorferi infection was confirmed by culture), 38% of baseline serum specimens had IgM immunoblot reactivty to P37 from strain B31. This frequency increased to 57% in convalescent serum samples collected 2-4 weeks after the beginning of antibiotic therapy. The specificity of the IgM response to P37 was 100%, as assessed with serum from healthy blood donors residing in non-endemic areas (OH and WY) (ASTPHLD supra).

The above studies indicated that the P37 antigen can be an important component in the criteria to interpret IgM immunoblots, augmenting OspC, BmpA (P39), and FlaB. The data in this report demonstrate the effective use of recombinant P37 as a test antigen for immunoassays. Reactivity of the recombinant protein was highly correlated with the immune responses to the natural product. Reactivity with recombinant P37 was seen with 32.5% of EM patients at their first visit to a physician. Sensitivity was 40% if weak reactions were included in the scoring.

When comparing reactivities of the serum samples with B. burgdorferi P37 vs. the recombinant P37 antigen, there were 3 discrepancies out of 61 tested serum samples. Of these, two recombinant negative samples gave only weak anti-P37 signals upon blotting against B. burdorferi whole cell lysate. It is possible that the weak signal scored as P37 in these blots may have been mistaken, as there were no P37 antibody standards to guide the blot scoring. It has been observed that BmpD, an antigen with a calculated molecular mass of 37,250 Da (Ramamoorthy R, 1996, “Molecular characterization, genomic arrangement, and expression of bmpD, a new member of the bmp class of genes encoding membrane proteins of Borrelia burgdorferi” Infect. Immun. 64: 1259-1264), migrated slightly faster than P37/FlaA in an 11.75% SDS-PAGE system, and recombinant BmpD did not react with P37-positive serum samples. Thus, it seems likely that there are other antigens in this size range that react weakly in B. burgdorferi whole cell immunoblots that could have been confused with the P37/FlaA. It should be noted that the P37-postive serum samples reacted prominently with the recombinant P37 at a 1:1000 dilution, whereas a positive P37 band was seen in the B. burgdorferi blots at a 1:100 dilution, suggesting greater sensitivity when using the recombinant P37 antigen.

One aspect that deserves scrutiny is potential cross-reactivity with FlaA's from other spirochete species. FlaA has been described in Treponema pallidum (Isaacs R D et al. 1990, “Expression in Escherichia coli of the 37-kilodalton endoflagellar sheath protein of Treponema pallidum by use of the polymerase chain reaction and a T7 expression system.” Infect. Immun. 58: 2025-2034), Spirocheata aurantia (Bramasha B et al., 1989 “Cloning and sequence analysis of flaA, a gene encoding a Spirocheata aurantia flagellar filament surface antigen.” J. Bacteriol. 171: 1692-1697). and Serpulina hyodysenteriae (Koopman M B H et al., 1992, “Cloning and DNA sequence analysis of a Serpulina (Trepoizenia) hyodysenteriae gene encoding a periplasmic flagellar sheath protein.” Infect. Immun. 60: 2920-2925), with antigenic cross-reactivity between the FlaA's of T. Pallidum, T. denticola, and T. phagedenis (Ge and Charon, 1997, J. Bacteriol. supra; Norris S J et al., 1988, “Antigenic relatedness and N-terminal sequence homology define two classes of major periplasmic flagellar proteins of Treponema pallidum subspecies pallidum and Treponema phagedenis.” J. Bacteriol. 170: 4072-4082). However no-crossreactivity was seen in the present study using four syphilis patient serum samples (FIG. 2).

The expression of full-length spirochetal FlaA's in E. coli has been shown to be difficult due to an apparent toxicity of the gene product to the cells (Ge and Charon, 1997, Infect. Immun. supra; Isaacs et al., 1990, supra). We were able to obtain expression of recombinant P37 antigen by using a construct of the gene minus the leader peptide (signal peptide). One study reported a failure to obtain FlaA expression using the vector pET-23a (Novagen) with or without the leader peptide. (Ge and Charon, 1997, Infect. Immunol. supra). In the present work, using a Novagen vector, pSCREEN, a pET derivative, we were successful in expressing large quantities of recombinant P37 antigen protein. This is due to the fact that we have discovered that for expression purposes, cell cultures must be started and induced from fresh transformants. Cultures begun from overnight starter cultures failed to produce appreciable amounts of expression product. Although no expression was demonstrated with the F1 and F3 constructs (FIG. 2), it was observed from colony immunoblot screenings of F1 and F3 transformants that a small percentage of the colonies were reactive against anti-P37 antibodies. Subsequent liquid culture growth of these positives, however, either failed to express any recombinant protein, or expressed small amounts of breakdown peptides. Thus it was determined that these constructs were genetically unstable past one or two propagations, which contrasted with the more stable F2 construct.

A recent report has stated that FlaA/P37 is not an immunodominant antigen associated with B. burdorferi infection, and therefore not a good candidate for the serological diagnosis of Lyme disease (Ge and Charon. 1997, Infect. Immun. supra). That conclusion was based upon Western blot analyses of 19 human serum samples from Lyme disease patients, and also a few samples from infected mice, rabbits, and monkeys. This teaching, which is contrary to the findings of the present invention, may be due to the use of serum samples from convalescent Lyme disease patients rather than early infection patients. Anti-P37 IgG does not occur as frequently as IgG antibodies of other specificities in late Lyme disease. However, the frequencies of anti-P37 IgG bands in immunoblots for patients with arthritis and late neurologic manifestations have been reported to be 44% and 48% respectively (Dressler F et al., 1993, “Western blotting in the serodiagnosis of Lyme disease.” J. Infect. Dis. 167: 392-400).

With the identification of this 37 kDa protein, described in previously published reports as a relevant antigen in serodiagnostic testing, as a product of the flaA gene, the P37 can be referred to as FlaA. The present invention demonstrates the use of the FlaA/P37 antigen for serodiagnosis of Lyme disease, in contrast to previously published reports that the FlaA protein is not suitable for serodiagnosis of Lyme disease. This P37/FlaA should not be confused with another B. burgdorferi 37 kDa protein described in a recent report as P37 (Fikrig E et al., 1997, “Borrelia burgdorferi P35 and P37 proteins, expressed in vivo, elicit protective immunity.” Immunity 6: 531-539), which is expressed in vivo only.

Diagnosis of the acute stage of Lyme disease has been difficult to support serologically because the current tests are relatively insensitive. The present invention demonstrates that FlaA detection can augment the set of recombinant molecules that are recognized early in the course of disease and contribute to the improved sensitivity of early testing for Lyme disease.

We claim:

1. A method for the diagnosis of Lyme Disease, the method comprising: contacting a sample to be tested with a recombinant FlaA protein, incubating for a sufficient time to allow formation of specific antibody-FlaA complexes, and detecting the antibody-FlaA complexes.

2. The method of claim 1 wherein said recombinant FlaA protein comprises a fusion protein.

3. The method of claim 2 wherein said fusion protein is an approximately 38 kDa T7 gene 10 product.

4. The method of claim 1, wherein the FlaA protein comprises an amino acid sequence as shown in SEQ ID NO:2.

5. The method of claim 4, wherein the FlaA protein comprises amino acids 1-319 of the amino acid sequence of SEQ ID NO:2.

6. The method of claim 1, wherein the FlaA protein comprises an amino acid sequence encoded by the nucleic acid sequence as shown in SEQ ID NO:3.

7. The method of claim 1, wherein the FlaA protein lacks a signal peptide.

8. The method of claim 1, wherein the FlaA protein is immobilized on a solid support.

9. The method of claim 1, wherein the FlaA protein further comprises a detectable label.

10. The method of claim 10, wherein the label is selected from the group consisting of a chemiluminescent label, a radioactive label, and a colorimetric label.

11. The method of claim 1, wherein the antibody-FlaA complex is detected by specific protein binding to the antibody specific for FlaA.

12. The method of claim 1, wherein the antibody is of the IgM subclass.

13. The method of claim 13, wherein the fusion partner of the FlaA fusion protein does not interfere with the antigenic epitopes of the FlaA protein.

14. The method of claim 1, wherein the steps are performed manually.

15. The method of claim 1, wherein the steps are automated.

16. A method for producing FlaA protein, the method comprising: providing freshly transformed host cells; constructing a DNA expression vector containing an expressible FlaA encoding DNA sequence; transforming a suitable host cell with said expression vector; plating out said transformed host cells; preparing large scale primary cell cultures from transformed host cells taken from a fresh transformant colony; and inducing FlaA protein expression from host cells in culture to produce a recombinant FlaA protein.

17. The method of claim 17, wherein the recombinant FlaA protein is encoded by a nucleic acid sequence as shown in SEQ ID NO:1.

18. The method of claim 17, wherein the FlaA protein comprises an amino acid sequence as shown in SEQ ID NO:2.

19. The method of claim 19, wherein the FlaA protein comprises amino acids 1-319 of the amino acid sequence of SEQ ID NO:2.

20. The method of claim 17, wherein the FlaA protein comprises an amino acid sequence encoded by the nucleic acid sequence as shown in SEQ ID NO:3.

21. The method of claim 17, wherein the FlaA protein is a fusion protein.

22. The method of claim 22, wherein the fusion protein comprises an approximately 38 kDa T7 gene 10 product.

23. The method of claim 17 wherein said transformed host cell is an E. coli cell.


SEQUENCE LISTING (6 sequences)

SEQ ID NO: 1 - DNA, 1655 bp, B.burgdorferi
  CDS 473..1498
  sig_peptide 473..538
  mat_peptide 539..1498
atgataatcttttttcaaaaaaggttttttattttcattctagcaagggatttgttgctaatttaagatatttaagagatgaacaaaatttgaaagataatttagatcttttagtaaaagattttcttttaggaagcaatgaggggttttcttttgggtttttattaagtgattcaagatttttatattcttttttaaagaatggagtttattatgtaaatctttcaagagaattttatgattcttttaataatggtgattataatgaatcttttgatgttaaggtcaatctttttgctatgtctttaataaaaacaatgcgctttaactatcctggtaagataaaaaagattattattcttgttgaagggtgtatcttaaaggagcaaagttgataaattaaattttactaataaaaataattaaaaaacgaaaattttataaaagatttatatataaggagttggtttacatgaaaaggaaagctaaaagtattttattttttttattatccactgttctttttgctcaagagactgatggattagcagagggttctaaaagggcagagcctggagaattagtcttagattttgccgagcttgcaagagatccaagttcaactagacttgatcttacaaattatgttgattatgtatattcgggcgcttctggtattgttaagccggaagatatggttgtagatcttgggataaataattggagcgttttacttactccttctgcaaggttgcaggcttacgttaaaaattcagttgttgcgcccgctgttgttaagagtgagtcaaaaaggtacgcaggtgatactattttgggggtaagagttttgtttccaagctattctcaatcatctgctatgattatgccaccatttaaaattcctttttattcaggggaaagtggcaatcaatttttaggcaaaggtcttattgataacattaaaaccatgaaagaaattaaggtatctgtttatagtttagggtatgagatagatcttgaggttttatttgaagatatgaatggcatggaatatgcttattctatgggtactttaaagtttaaagggtgggctgatttaatttggtcaaatcctaactatattcctaatatatcatccagaattattaaagacgatgttccaaattatcctcttgcttcaagtaaaatgagatttaaggcttttagagtttcaaagtcacacagttcaaaagttaaaaatttcatcttttatgttaaagatttaagagttctttatgataagctaagtgtttcaatagattctgatattgacagtgagtctgtatttaaagtttatgagactagcggaactgaatcccttcgtaaattaaaggcacacgaaacttttaaaagagttttaaagcttagagaaaaaatttctatcgctgaaggctctttccaaaactttgtagaaaagattgagagtgaaaaacctgaagaatcatctccgaaaaattaggtttaaattaatatgtaaagctacctaaaaggtttgctttacatattaaaataataggaaatagtatatggaaatattagatttggaaaatgaagagcttttaggagttttttttgaagaagctcaaaatcttgtagatatccttgaagagaatatt

SEQ ID NO: 2 - protein, 341 aa, B.burgdorferi
MKRKAKSILFFLLSTVLFAQETDGLAEGSKRAEPGELVLDFAELARDPSSTRLDLTNYVDYVYSGASGIVKPEDMVVDLGINNWSVLLTPSARLQAYVKNSVVAPAVVKSESKRYAGDTILGVRVLFPSYSQSSAMIMPPFKIPFYSGESGNQFLGKGLIDNIKTMKEIKVSVYSLGYEIDLEVLFEDMNGMEYAYSMGTLKFKGWADLIWSNPNYIPNISSRIIKDDVPNYPLASSKMRFKAFRVSKSHSSKVKNFIFYVKDLRVLYDKLSVSIDSDIDSESVFKVYETSGTESLRKLKAHETFKRVLKLREKISIAEGSFQNFVEKIESEKPEESSPKN

SEQ ID NO: 3 - DNA, 21 bp, B.burgdorferi
atgaaaaggaaagctaaaagt

SEQ ID NO: 4 - DNA, 19 bp, B.burgdorferi
gatggattagcagagggtt

SEQ ID NO: 5 - DNA, 21 bp, B.burgdorferi
tgggataaataattggagcgt

SEQ ID NO: 6 - DNA, 21 bp, B.burgdorferi
ctaatttttcggagatgattc